(19) United States
(12) Patent Application Publication
(10) Pub. No.: US 2011/0152495 A1
(43) Pub. Date: Jun. 23, 2011

(54) METHOD FOR MOULDING POLY(1,4-DIOXANONE)

(75) Inventors: Philippe Le Goff, Epalinges (CH); Raymond Andrieu, Yens (CH)

(73) Assignee: BIORING SA, Lonay (CH)

(21) Appl. No.: 13/001,865

(22) PCT Filed: Jun. 30, 2008
(86) PCT No.: PCT/FR2008/051199

§ 371 Date: Mar. 15, 2011

(51) Int. Cl. A61L 31/06; B29C 45/72; C08G 63/664

(52) U.S. Cl. 528/354; 264/328.14

(57) ABSTRACT

A method of moulding a bioresorbable polymer for producing a bioresorbable medical device includes the following successive steps: (a) heating a poly(1,4-dioxanone) in the absence of any solvent of this polymer to a mass temperature between 145° C. and 165° C., (b) injection moulding the molten mass obtained in step (a) in a mould which is at a temperature of 80° C. to 115° C. lower than the mass temperature of the poly(1,4-dioxanone), (c) cooling the mould until solidification of the mass of poly(1,4-dioxanone), and (d) removing the thus obtained part from the mould. Also described is a moulded part which can be obtained by such a method as well as a medical device containing such a part.

The present invention relates to a new method of moulding poly(1,4-dioxanone) for producing a bioresorbable medical device, as well as a moulded part and a medical device which can be obtained using such a method.

For close to 50 years, interest in the medical field for surgical implants based on non-permanent materials which are intended to disappear over time has only increased.

These materials are generally called “biodegradable”, “bioerodible”, “bioabsorbable” or “bioresorbable”. Within the scope of this application, we will preferentially use the term “bioresorbable” or “biodegradable”.

In the case of biodegradable implants, the question of the safety of the degradation products of the material is raised in the sense where all of the material constituting the implant or the medical device is released in the body of the patient in order to be converted and/or metabolised thereby.

Among the biodegradable polymer materials, a distinction is generally made between biological materials of natural origin, such as collagen or cellulose, and synthetic polymers. However, the variety of available biodegradable polymers compatible with a medical use today still remains relatively limited which restricts more or less directly the range of mechanical properties of the medical devices able to be made therefrom.

During the last two decades, a large number of polymer structures which are potentially biodegradable by a hydrolysis process have been proposed without any of them achieving a stage of development sufficient for them to be considered for use in the field of medical devices.

In this context, at the beginning of the 2000s five polymers were principally approved by the Federal Drug Administration (FDA) for use in implants. These five polymers are polylactic acid, polyglycolic acid, polydioxanones, polycaprolactones and polyanhydrides. Of course, copolymers containing two or more types of monomers constituting these homopolymers have a degree of safety comparable therewith.

Polydioxanones and in particular poly(1,4-dioxanone) are known to degrade by hydrolysis without producing toxic degradation products. Polydioxanone is further advantageous in that it degrades in vivo solely by a hydrolysis process, in other words the degradation kinetics of polydioxanone is not modified by enzymatic processes.

However, applications for medical devices based on polydioxanone remain limited since this polymer is difficult to use and is reputed to yield, after being shaped, materials with mediocre mechanical properties.

U.S. Pat. No. 4,490,326 proposes a polydioxanone injection moulding process for producing surgical devices which are bioresorbable and implantable and have a satisfactory combination of mechanical properties (mechanical strength, toughness, flexibility, functional integrity). This document recommends the injection moulding of polydioxanone from a molten mass having a temperature as close as possible to the melting temperature of the polydioxanone (M_{P}=109-110° C.). Thus, patent U.S. Pat. No. 4,490,326 describes that the polydioxanone which has been previously melted in an extruder, having a temperature between 110° C. and 140° C., preferably between 110° C. and 115° C., is injected into a mould maintained at a temperature at most equal to 35° C. and is maintained under pressure during a period of time sufficient to obtain the total or partial hardening of the part before being removed from the mould.

Whilst it is true that such a moulding method enables moulded parts to be obtained which have, when removed from the mould, a rather satisfactory combination of mechanical properties, it is also important that these properties last for a sufficiently long period of time, in particular during the storing of the moulded part in order to be able to envisage an application on an industrial scale. Now, the Applicant has observed that the manufacturing conditions for the moulded parts had a notable influence on these mechanical properties which unfortunately were not maintained over time. At the end of a period of storage at ambient temperature of about one month, the moulded parts became brittle and friable. Their mechanical strength became insufficient thus preventing their use as bioresorbable implants. It will be easily understood that this non-durability of the mechanical properties of the moulded parts is a considerable disadvantage in terms of the commercialisation of implantable medical devices based on polydioxanone.

Within the scope of his research aiming to perfect new implantable medical devices based on bioresorbable polymer materials, the Applicant has unexpectedly found that, contrary to the teaching of patent U.S. Pat. No. 4,490,326, moulding polydioxanone at temperatures above those recommended in this document enabled moulded parts to be obtained which not only had satisfactory mechanical properties but which advantageously retained these properties for several months.

The Applicant has further noted that in order to obtain favourable mechanical properties from a molten mass of polydioxanone heated to a relatively high temperature, i.e., about 145° C. to 165° C., it was important to not cool the molten mass too quickly in order to obtain a good degree of crystallinity of the polydioxanone.

The object of the present invention is therefore a method of moulding a bioresorbable polymer for producing a bioresorbable medical device, comprising the following successive steps:

(a) heating a poly(1,4-dioxanone) in the absence of any solvent of this polymer to a mass temperature between 145° C. and 165° C.,
(b) injection moulding the molten mass obtained in step (a) in a mould which is at a temperature of 80° C. to 115° C. lower than the mass temperature of the poly(1,4-dioxanone),
(c) cooling the mould until solidification of the mass of poly(1,4-dioxanone), and
(d) removing the thus obtained part from the mould.

The term “mass temperature” used in the present invention designates the temperature of the polydioxanone measured using a thermometer at the centre of the molten mass. This mass temperature is lower than the set temperature of the heating device and also lower than the temperature of the crucible used to melt the polymer.

Within the scope of routine tests performed at different temperatures, the Applicant has noted that it was possible to modify the mechanical properties of the obtained moulded polymer materials by appropriately selecting the mass temperature in step (a): temperatures in the upper half of the claimed range thus lead to rather rigid moulded polymer materials whilst heating temperatures in the lower half of this range yield rather flexible materials.

Consequently, in one embodiment of the method of the invention, when obtaining flexible poly(1,4-dioxanone) materials, the poly(1,4-dioxanone) is heated in step (a) to a mass temperature between 145° C. and 155° C.

In contrast, in another embodiment of the method, when preparing materials which are relatively more rigid, the poly(1,4-dioxanone) is heated in step (a) to a mass temperature between 155° C. and 165° C.

Generally, the most favourable moulded materials are those prepared from a molten mass heated to a mass temperature close to 155° C., in other words the mass temperature of the poly(1,4-dioxanone) in step (a) is preferably between 148° C. and 162° C., in particular between 152° C. and 158° C., and ideally between 154° C. and 156° C.

In order to prevent possible thermal degradations of the polydioxanone, it is desirable that the heating step (step (a)) of the method of the invention lasts for a relatively short period of time, preferably all the shorter the higher the mass temperature. Generally, the total duration of heating step (a), including the temperature-increasing phase and the temperature-maintaining phase prior to moulding, is lower than 60 minutes, preferably lower than 45 minutes, in particular between 10 and 30 minutes.

The polydioxanone can be melted for example in a crucible placed on an electric heating plate. Taking into account the high viscosity of the molten mass of the polydioxanone, this melting step is preferably effected in the absence of mechanical stirring.

The poly(1,4-dioxanone) used in the method of the present invention preferably has a relatively low molecular mass which is such that its inherent viscosity, measured in 0.1 wt. % solution in hexafluoroisopropanol (HFIP) at a temperature of 30° C., is between 1.1 and 1.8 dl/g and is preferably between 1.2 and 1.6 dl/g. This inherent viscosity range, and thus this molecular mass of the polymer, is preferred owing to the good mechanical properties conferred on the obtained moulded parts and to the behaviour compatible with the thermal and kinetic stresses of the moulding method of the present invention.

As indicated previously, the temperature of the mould, in which the molten mass of poly(1,4-dioxanone) is injected, is 80° C. to 115° C. lower than that of the mass temperature of the molten polymer. This temperature difference between the injected polymer and the mould ensures a good surface appearance of the obtained part and a crystallisation sufficient to provide the part with a satisfactory mechanical strength. In one preferred embodiment of the method of the invention, the temperature of the mould in step (b) is 85° C. to 105° C. lower, preferably 90 to 100° C. lower, than the mass temperature of the poly(1,4-dioxanone) of step (a).

The molten polymer can be introduced into the mould for example by injecting the molten contents into the receiving mould. To this end, the base of the crucible is made to be movable so as to permit the injection of the molten material by a piston effect caused by the pressure exerted by the cylinder of a hydraulic press on the external face of the movable base of the crucible. The crucible, in combination with an appropriate funnel fixed to the mould, thus acts as an injection syringe for the molten material.

It is highly recommended to maintain the molten mass, after being injected into the mould, at the injection pressure for a certain period of time.

The period of time for maintaining the molten mass of polydioxanone under pressure in the mould depends upon the size and geometry of the part, upon the temperature of the mould and upon the cooling rate thereof in step (c). Experience shows that a period of time between 5 seconds and 40 seconds is suitable and that a period of time between 10 and 20 seconds is particularly advantageous.

After injecting the molten poly(1,4-dioxanone), the mould—initially at the temperature indicated above—is slowly cooled. This cooling can be effected simply by stopping the heating and dissipating the heat or even by actively cooling the mould, for example by way of contact with a cold surface.

The total duration of the cooling phase (step (c)) is preferably between 1 and 30 minutes, in particular between 2 and 10 minutes.

The hardened poly(1,4-dioxanone) part is preferably removed from the mould in step (d) only when its surface temperature has reached a value lower than 50° C., preferably between ambient temperature and 45° C.

Another object of the present invention is a moulded poly(1,4-dioxanone) part which can be obtained by the method described above. The poly(1,4-dioxanone) parts obtained in accordance with the method of the present invention have, in fact, properties different from those of moulded polydioxanone parts prepared in accordance with the Prior Art. They are characterised in particular by a greater stability of the mechanical properties over time. The poly(1,4-dioxanone) parts obtained in accordance with the method of the present invention do not become brittle at the end of only one month and can be stored, before being implanted, for at least 12 months, generally at least 24 months from the time of being removed from the mould.

Finally, yet another object of the present invention is a medical device formed from such a moulded poly(1,4-dioxanone) part, produced therefrom for example by a shaping process, and/or containing at least one such part.

### Example

3.0 g of poly(1,4-dioxanone) (inherent viscosity 1.4 dl/g at 30° C. in HFIP) is introduced into a melt crucible having a movable base and the crucible containing the polymer is placed on a heating plate previously set to about 220° C. A thermometer is introduced into the centre of the molten mass. In parallel therewith, a mould is set to a temperature between 50 and 60° C.

When the mass temperature of the poly(dioxanone) measured using the thermometer reaches about 148 to 152° C., and making sure that the heating period does not exceed 30 minutes, the crucible is placed upside-down on the funnel of the mould and the cylinder of a hydraulic press is actuated so as to inject the molten polydioxanone into the mould. The pressure of the cylinder on the movable base of the crucible is maintained for about 15 seconds.

The cylinder is then lifted off and the crucible is removed from the mould. The mould is cooled by placing it for about 5 minutes on a plate maintained at ambient temperature. The mould is then opened and the moulded part is extracted using Brussels forceps.

1. Method of moulding a bioresorbable polymer for producing a bioresorbable medical device, comprising the following successive steps:
(a) heating a poly(1,4-dioxanone) in the absence of any solvent of this polymer to a mass temperature between 145° C. and 165° C.,
(b) injection moulding the molten mass obtained in step (a) in a mould which is at a temperature of 80° C. to 115° C. lower than the mass temperature of the poly(1,4-dioxanone),
(c) cooling the mould until solidification of the mass of poly(1,4-dioxanone), and
(d) removing the thus obtained part from the mould.

2. Moulding method as claimed in claim 1, characterised in that the poly(1,4-dioxanone) is heated in step (a) to a mass temperature between 148° C. and 162° C., preferably between 152° C. and 158° C., and in particular between 154° C. and 156° C.

3. Moulding method as claimed in claim 1, characterised in that the poly(1,4-dioxanone) is heated in step (a) to a mass temperature between 145° C. and 155° C.

4. Moulding method as claimed in claim 1, characterised in that the poly(1,4-dioxanone) is heated in step (a) to a mass temperature between 155° C. and 165° C.

5. Moulding method as claimed in claim 1, characterised in that the temperature of the mould in step (b) is 85° C. to 105° C. lower, preferably 90 to 100° C. lower, than the mass temperature of the poly(1,4-dioxanone) of step (a).

6. Moulding method as claimed in claim 1, characterised in that the poly(1,4-dioxanone) has a molecular mass such that its inherent viscosity, measured in 0.1 wt. % solution in hexafluoroisopropanol (HFIP) at a temperature of 30° C., is between 1.1 and 1.8 dl/g and is preferably between 1.2 and 1.6 dl/g.

7. Moulding method as claimed in claim 1, characterised in that the total duration of heating step (a) is lower than 60 minutes, preferably lower than 45 minutes, in particular between 10 and 30 minutes.

8. Moulding method as claimed in claim 1, characterised in that the duration of the cooling step (step (c)) is between 1 and 30 minutes, preferably between 2 and 10 minutes.

9. Moulding method as claimed in claim 1, characterised in that the poly(1,4-dioxanone) part is removed from the mould (step (d)) when its surface temperature of the part is lower than 50° C., preferably between ambient temperature and 45° C.

10. Moulded poly(1,4-dioxanone) part which can be obtained by the method as claimed in claim 1.

11. Medical device formed from the moulded part as claimed in claim 10, made therefrom and/or containing same.

12. Moulding method as claimed in claim 2, characterised in that the temperature of the mould in step (b) is 85° C. to 105° C. lower, preferably 90 to 100° C. lower, than the mass temperature of the poly(1,4-dioxanone) of step (a).

13. Moulding method as claimed in claim 3, characterised in that the temperature of the mould in step (b) is 85° C. to 105° C. lower, preferably 90 to 100° C. lower, than the mass temperature of the poly(1,4-dioxanone) of step (a).

14. Moulding method as claimed in claim 4, characterised in that the temperature of the mould in step (b) is 85° C. to 105° C. lower, preferably 90 to 100° C. lower, than the mass temperature of the poly(1,4-dioxanone) of step (a).

15. Moulding method as claimed in claim 2, characterised in that the poly(1,4-dioxanone) has a molecular mass such that its inherent viscosity, measured in 0.1 wt. % solution in hexafluoroisopropanol (HFIP) at a temperature of 30° C., is between 1.1 and 1.8 dlg and is preferably between 1.2 and 1.6 dl/g.

16. Moulding method as claimed in claim 3, characterised in that the poly(1,4-dioxanone) has a molecular mass such that its inherent viscosity, measured in 0.1 wt. % solution in hexafluoroisopropanol (HFIP) at a temperature of 30° C., is between 1.1 and 1.8 dl/g and is preferably between 1.2 and 1.6 dl/g.

17. Moulding method as claimed in claim 4, characterised in that the poly(1,4-dioxanone) has a molecular mass such that its inherent viscosity, measured in 0.1 wt. % solution in hexafluoroisopropanol (HFIP) at a temperature of 30° C., is between 1.1 and 1.8 dl/g and is preferably between 1.2 and 1.6 dl/g.

18. Moulding method as claimed in claim 5, characterised in that the poly(1,4-dioxanone) has a molecular mass such that its inherent viscosity, measured in 0.1 wt. % solution in hexafluoroisopropanol (HFIP) at a temperature of 30° C., is between 1.1 and 1.8 dl/g and is preferably between 1.2 and 1.6 dl/g.

19. Moulding method as claimed in claim 2, characterised in that the total duration of heating step (a) is lower than 60 minutes, preferably lower than 45 minutes, in particular between 10 and 30 minutes.

20. Moulding method as claimed in claim 3, characterised in that the total duration of heating step (a) is lower than 60 minutes, preferably lower than 45 minutes, in particular between 10 and 30 minutes.
